(19) United States
(12) Patent Application Publication
(10) Pub. No.: US 2025/0281179 A1
(43) Pub. Date: Sep. 11, 2025

(54) STAPLE HEAD AND SURGICAL STAPLER

(71) Applicant: TOUCHSTONE INTERNATIONAL MEDICAL SCIENCE CO., LTD., Suzhou (CN)

(72) Inventors: Hua ZANG, Suzhou (CN); Yanping YE, Suzhou (CN); Kaifen FU, Suzhou (CN); Yujin MIAO, Suzhou (CN)

(73) Assignee: TOUCHSTONE INTERNATIONAL MEDICAL SCIENCE CO., LTD., Suzhou (CN)

(21) Appl. No.: 19/218,822

(22) Filed: May 27, 2025

Related U.S. Application Data
(63) Continuation of application No. PCT/CN2023/134235, filed on Nov. 27, 2023.

(30) Foreign Application Priority Data
Dec. 8, 2022 (CN) 202211570672.7
Dec. 8, 2022 (CN) 202223294093.2

(51) Int. Cl. A61B 17/072; A61B 17/00; A61B 90/00

(52) U.S. Cl. CPC A61B 17/07207; A61B 90/03; A61B 2017/00477; A61B 2017/00592; A61B 2017/07214; A61B 2017/07285; A61B 2090/035

(57) ABSTRACT

A staple head includes a first jaw and a second jaw, a connecting member, a stop member and a firing mechanism. The connecting member is connected to proximal ends of the first jaw and the second jaw. The stop member is rotatably mounted on the connecting member and includes a first blocking portion. The firing mechanism is movable along an axial direction of the stapler and includes a second blocking portion. In a first state, the first blocking portion is located on a path where the second blocking portion moves in a direction toward a distal end side. When an external force is applied to the stop member, the stop member rotates relative to the connecting member in a first direction such that the first blocking portion moves in a direction away from an axial center of the stapler, the stop member enters a second state.

### CROSS-REFERENCE TO RELATED APPLICATIONS

The present application is a continuation application of International Application No. PCT/CN2023/134235, filed on Nov. 27, 2023, which claims priority to Chinese Patent Application No. 202211570672.7, filed on Dec. 8, 2022, and Chinese Patent Application No. 202223294093.2, filed on Dec. 8, 2022. All of the aforementioned applications are hereby incorporated by reference in their entireties.

### TECHNICAL FIELD

The present application relates to the field of medical instruments technologies, and in particular, to a staple head and a surgical stapler.

### BACKGROUND

In the conventional technologies, a surgical stapler includes a stapler body, an operating handle movably connected to the stapler body, and a staple head cooperating with the body. A transmission mechanism is disposed inside the stapler body. The staple head includes a staple cartridge assembly and a staple anvil assembly arranged opposite to each other, and a cutter movable within the staple head. A firing block is provided inside the staple cartridge assembly. During surgery, a tissue is placed between the nail anvil assembly and the staple cartridge assembly, and a distance between the staple anvil assembly and the staple cartridge assembly is adjusted to gradually clamp the tissue. The operating handle is then held to make the transmission mechanism drive the cutter to move toward a distal end, then the cutter propels the firing block to move to make a staple be ejected and formed on a staple anvil while cutting the tissue simultaneously, and thus anastomosis and cutting of the tissue is completed.

For conventional staplers, after one time of firing is completed, all staples in a staple cartridge have been stapled into the tissue, so that the staple cartridge is required to be replaced before the next firing. Before replacing the staple cartridge, if a surgeon, due to inexperience or carelessness, holds the operating handle again, the cutter will be driven to move toward the distal end once more through the transmission mechanism, thereby resulting in the tissue being cut without being stapled, and thus leading to surgical failure.

### SUMMARY

In view of the problem in the conventional technologies, a purpose of the present application is to provide a staple head and a surgical stapler. When a stop member is in a first state, it blocks a firing mechanism to move toward a distal end, so that accidental firing of the stapler is effectively avoided. Meanwhile, when the stop member is in a second state, it does not block the firing mechanism to move, so that normal firing of the stapler is allowed.

An embodiment of the present application provides a staple head, including:
a first jaw and a second jaw arranged opposite to each other;
a connecting member, connected to proximal ends of the first jaw and the second jaw;
a stop member rotatably mounted on the connecting member and including a first blocking portion; and
a firing mechanism, movable along an axial direction of the surgical stapler and including a second blocking portion; where
when the stop member is in a first state, the first blocking portion is located on a path where the second blocking portion moves in a direction toward a distal end; and when an external force is applied to the stop member, the stop member rotates relative to the connecting member in a first direction such that the first blocking portion moves away from a axial center of the stapler, the stop member enters a second state, and the first blocking portion disengages from the path where the second blocking portion moves toward the distal end.

In some embodiments, a distal end of the connecting member is provided with a mounting portion for mounting the stop member; when the first jaw and the second jaw are in an open state, the mounting portion is at least partially located on a side of the second blocking portion away from an axial center of the stapler; and the first blocking portion and the second blocking portion are provided with a first distance along the axial direction of the surgical stapler.

In some embodiments, the stop member also includes a pivoting portion rotatably mounted on the mounting portion via a pin shaft; and when the stop member is in the first state, the first blocking portion protrudes in a direction toward the axial center direction of the stapler relative to the pivoting portion.

In some embodiments, the staple head also includes a driving member disposed on the first jaw and movable along the axial direction of the stapler, wherein the first jaw is rotatable relative to the connecting member. When the driving member is located at the proximal end of the first jaw and the first jaw rotates to be relatively closed with the second jaw, the driving member abuts against the stop member and drives the stop member to enter the second state from the first state.

In some embodiments, when the stop member enters the second state from the first state, the firing mechanism moves toward the distal end for a second distance, and the first distance is greater than or equal to the second distance.

In some embodiments, a first driving portion is provided on a proximal end of the driving member, and a second driving portion cooperating with the first driving portion is provided on a side of the stop member facing toward the axial center of the stapler.

In some embodiments, a side of the first driving portion opposite to the second driving portion is provided with a first guiding surface, and a side of the second driving portion opposite to the first driving portion is provided with a second guiding surface.

In some embodiments, the first blocking portion and the second driving portion are integrally formed; and when the stop member is in the first state, a side of the first blocking portion facing toward the first jaw is provided with the second guiding surface.

In some embodiments, the firing mechanism includes a cutter and a cutter push rod, a distal end of the cutter is provided with the second blocking portion, and a proximal end of the cutter is connected to the cutter push rod.

In some embodiments, when the stop member is in the second state and the firing mechanism moves toward the distal end, the stop member at least partially abuts against a side surface of the cutter push rod to be remained in the second state.

In some embodiments, the staple head also includes an elastic member configured to apply a biasing force to the stop member to drive the stop member to rotate in a second direction opposite to the first direction.

In some embodiments, the mounting portion is provided with a mounting groove, and an opening of the mounting groove faces toward the axial center of the stapler, and the stop member at least partially enters into the mounting groove.

In some embodiments, the elastic member is a torsion spring disposed at the pin shaft or a compression spring disposed between the stop member and an inner wall of the mounting groove.

In some embodiments, the connecting member includes a first limiting portion, and the mounting groove is provided with a second limiting portion; and the first limiting portion cooperates with the second limiting portion to limit a rotation angle of the connecting member in the second direction; and/or, the connecting member also includes a third limiting portion, and the stop member includes a fourth limiting portion; and the third limiting portion cooperates with the fourth limiting portion to limit a rotation angle of the connecting member in the first direction.

In some embodiments, a compression spring channel is provided inside the mounting groove, the compression spring is movably disposed in the compression spring channel, the compression spring channel is arranged perpendicular to a side wall of the firing mechanism, and an axial width of the compression spring channel is greater than an axial width of the mounting groove.

The embodiments of the present application also provide a surgical stapler including the foregoing staple head.

The staple head and surgical stapler provided in the present application have the following advantages.

By adopting the technical solution provided in the present application, when the stapler satisfies a firing condition, the stop member enters the second state and does not block the firing mechanism to move, so that the firing mechanism may be driven to move toward the distal end to normally fire the stapler. After firing of the stapler is completed, the stop member enters the first state. By the first blocking portion cooperating with the second blocking portion, the movement of the firing mechanism toward the distal end is effectively blocked, thereby preventing accidental secondary firing of the stapler, and thus surgical effect is effectively improved.

### BRIEF DESCRIPTION OF THE DRAWINGS

The other features, purposes, and advantages of the present application will become more apparent through the detailed description of non-limiting embodiments with reference to the following drawings.

FIG. 1 is a schematic diagram showing cooperation between a staple head and a sleeve according to an embodiment of the present application.

FIG. 2 is a schematic diagram showing cooperation between a staple head and a sleeve according to another embodiment of the present application.

FIG. 3 is a schematic diagram showing cooperation between a staple head and a sleeve with a staple anvil housing being omitted according to an embodiment of the present application.

FIG. 4 is a top view of a staple head according to an embodiment.

FIG. 5 is a cross-sectional view taken along a line A-A in FIG. 4.

FIG. 6 is a schematic diagram showing cooperation between a connecting member and a stop member according to an embodiment of the present application.

FIG. 7 is a schematic diagram showing cooperation between a connecting member and a stop member according to another embodiment of the present application.

FIG. 8 is a schematic structural diagram of a stop member according to an embodiment of the present application.

FIG. 9 is a schematic structural diagram of a connecting member according to an embodiment of the present application.

FIG. 10 is a schematic diagram showing cooperation between a stop member in a first state and a firing mechanism according to an embodiment of the present application.

FIG. 11 is a schematic diagram showing cooperation between a stop member and a cutter when the stop member is in the first state according to an embodiment of the present application.

FIG. 12 is a schematic diagram showing cooperation between a stop member and a firing mechanism after a stapler is closed according to an embodiment of the present application.

FIG. 13 is a schematic structural diagram of a driving member according to an embodiment of the present application.

FIG. 14 is a schematic diagram showing cooperation between a driving member and a stop member according to an embodiment of the present application.

FIG. 15 is a schematic diagram showing cooperation between a stop member and a cutter when the stop member is in a second state according to an embodiment of the present application.

FIG. 16 is a schematic diagram showing cooperation between a stop member and a firing mechanism during a firing process of the stapler according to an embodiment of the present application.

FIG. 17 is a schematic diagram showing cooperation between a stop member and a firing mechanism after the stapler is fired according to an embodiment of the present application.

FIG. 18 is a schematic diagram showing cooperation between a stop member and a connecting member according to an embodiment of the present application.

FIG. 19 is a schematic structural diagram of a connecting member according to an embodiment of the present application.

FIG. 20 is a cross-sectional view taken along a line B-B in FIG. 18.

### DETAILED DESCRIPTION OF THE EMBODIMENTS

Exemplary embodiments will be described comprehensively with reference to the drawings. It should be understood that, the exemplary embodiments may be implemented in various forms and are not to be construed as being limited to the embodiments set forth herein. On the contrary, these embodiments are provided to make the present application be comprehensive and complete, and to fully convey the concepts of the exemplary embodiments to persons skilled in the art. In the drawings, the same reference numerals denote the same or similar structures, and thus redundant descriptions thereof will be omitted. The terms “or” and “either” in the specification may be interpreted to mean either “and” or “or” respectively. Although terms such as “upper,” “lower,” and “between” may be used in the specification to describe various exemplary features and elements of the present application, such terms are used herein merely for convenience of description based on the orientations shown in the drawings. Nothing in the present specification shall be interpreted as requiring a particular three-dimensional orientation of a structure to fall within the scope of the present application. Although terms such as “first” or “second” is used herein for denoting certain features, such terms are merely used for illustrative purposes and are not to be construed as limiting the quantity and importance of the specific features.

In view of the problem in the related technologies, it is necessary to provide a structure that prevents accidental secondary firing when the staple cartridge has not been replaced after one time of the firing.

The present application provides a staple head for a surgical stapler and a surgical stapler including the staple head. The surgical stapler includes a stapler body, an operating handle and a staple head disposed at a distal end of the stapler body. When a firing condition of the stapler is satisfied, the operating handle is held for firing to make an actuation force be transmitted to the staple head through a transmission mechanism inside the stapler body, and thereby enabling the staple head to complete anastomosis and cutting operations of a tissue. The staple head includes a first jaw and a second jaw arranged opposite to each other, a connecting member, a stop member and a firing mechanism. The connecting member is connected to the proximal end of the first jaw and the second jaw. The stop member is rotatably mounted on the connecting member and includes a first blocking portion. The firing mechanism is movable along an axial direction of the stapler and includes a second blocking portion.

When the stop member is in a first state, the first blocking portion is located on a path where the second blocking portion moves in a direction toward a distal end, so that movement of the firing mechanism toward the distal end is blocked, thereby the stapler cannot be fired, and thus accidental firing of the stapler is effectively avoided. When an external force is applied to the stop member, the stop member rotates relative to the connecting member in a first direction such that the first blocking portion moves away from an axial center of the stapler, the stop member enters a second state to make the first blocking portion disengage from the path that the second blocking portion moves toward the distal end. In this point, the firing mechanism is driven to move toward the distal end, and thereby normally firing the stapler.

Structures of staple heads in each specific embodiment of the present application are described in detail below with reference to the drawings. It may be understood that, the specific embodiments are not intended to limit the scope of protection of the present application.

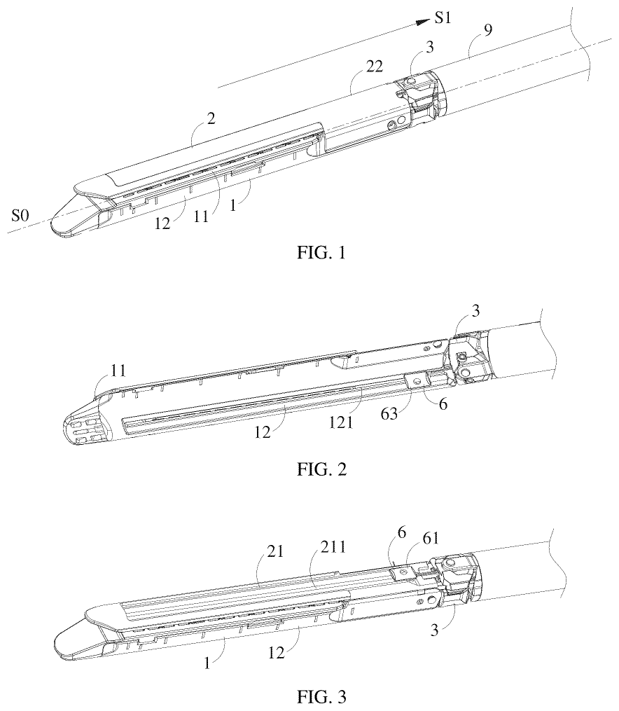

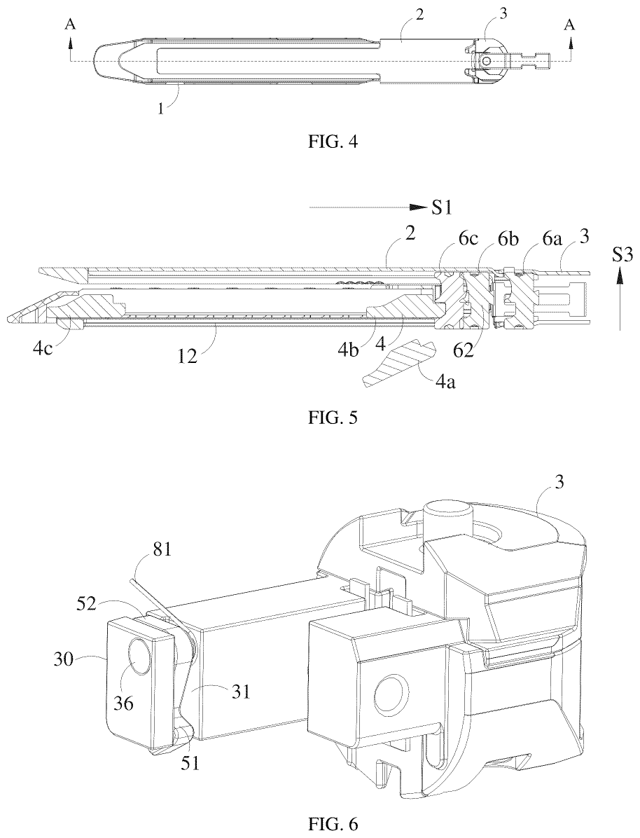

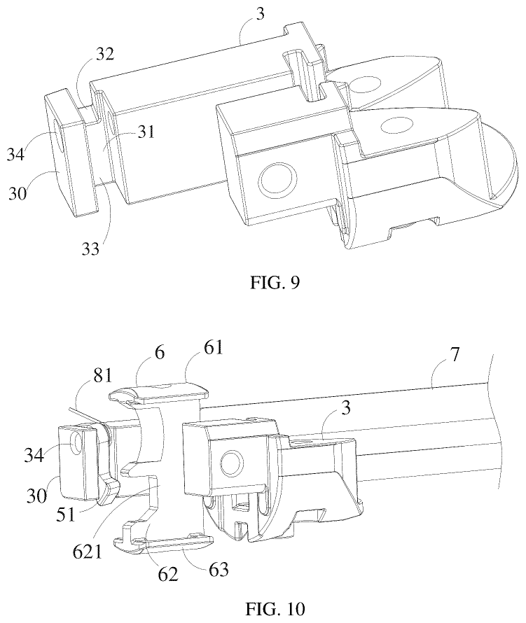

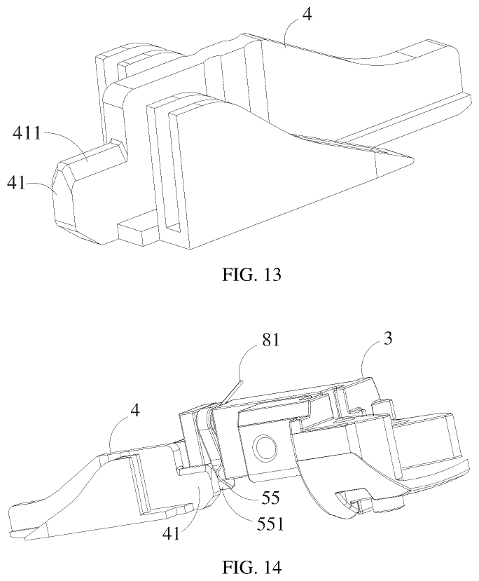

FIG. 1 to FIG. 17 show a structure of a staple head according to a first embodiment of the present application. As shown in FIG. 1 to FIG. 5, the staple head includes a first jaw and a second jaw arranged opposite to each other, and a connecting member 3. The staple head is disposed on a distal end of a sleeve 9 of a stapler body. A jaw opening is formed between the first jaw and the second jaw, in general, one of the first jaw and the second jaw is in a fixed state, and the other may be in a relatively opening state or a relatively closed state relative to the fixed jaw. In this embodiment, take the second jaw serving as the fixed jaw, the first jaw being movable, the first jaw being provided with a staple cartridge assembly 1, and the second jaw being provided with a staple anvil assembly 2 as an example for description. The staple cartridge assembly 1 includes a staple cartridge frame 12 and a staple cartridge 11 for accommodating staples therein. The staple anvil assembly 2 includes a staple anvil housing 22 and a staple anvil body 21. Proximal ends of the staple cartridge assembly 1 and the staple anvil assembly 2 are connected to the connecting member 3, and the staple cartridge assembly 1 is rotatable relative to the connecting member 3, so that the staple cartridge assembly 1 has an open state and a closed state relative to the staple anvil assembly 2, that is, the staple head has an opening state and a closed state of the jaw opening.

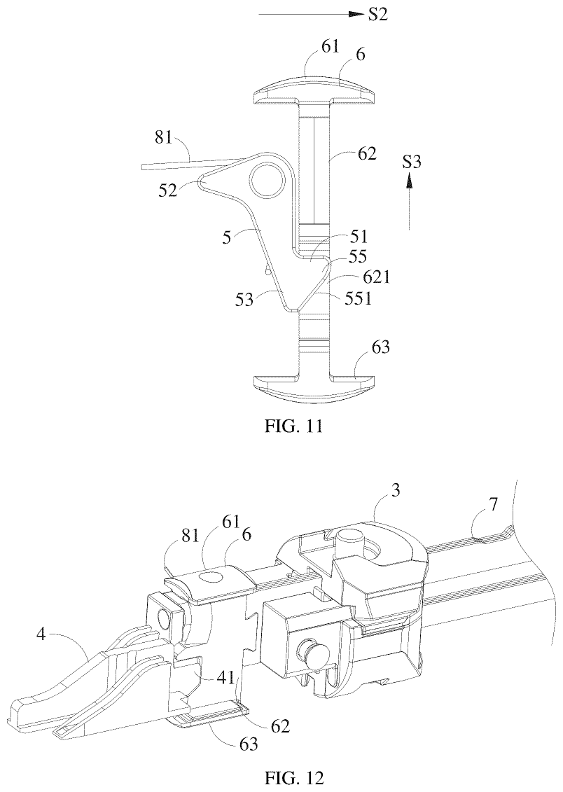

In the present application, a distal end and a proximal end are defined relative to an operator, the proximal end refers to a side closer to the operator, and the distal end refers to a side farther from the operator, that is, a side closer to a surgical position is a distal end. A direction along an axial center of the stapler is defined as an axial direction, that is, a direction from a distal end to a proximal end side of the stapler, or a direction from the proximal end side to the distal end of the stapler. A direction S1 in FIG. 1 is defined as the direction from the distal end to the proximal end side of the stapler, the direction S1 or a direction opposite to the direction S1 is the axial direction. In FIG. 5, a direction S3 is a longitudinal direction, that is, a height direction. In FIG. 11, a direction S2 is transverse direction, that is, a width direction. In FIG. 1, a direction S0 is the axial center direction of the stapler. For a component, a side of the component close to an axial center of the stapler is an inner side, and a side away from the axial center of the stapler is an outer side.

The staple head also includes a firing mechanism, and the firing mechanism includes a cutter 6 and a cutter push rod 7. A distal end of the cutter push rod 7 is connected to a proximal end of the cutter 6, and the cutter push rod 7 may be driven to drive the cutter 6 move along an axial direction of the stapler. The cutter 6 includes a first crossbeam 61, a connecting portion 62 and a second crossbeam 63 that are arranged sequentially from top to bottom, and an upper distal end of the connecting portion 62 is a blade for cutting a tissue. Two ends of the connecting portion 62 enters a first cutter groove 211 of the staple anvil body 21 and a second cutter groove 121 of the staple cartridge frame 12, respectively, and the first crossbeam 61 and the second crossbeam 63 respectively cooperate with the staple anvil body 21 and the staple cartridge frame 12. As shown in FIG. 5, when the cutter 6 is located on the proximal end, it has three positions, namely a position 6a, a position 6b, and a position 6c. When the staple cartridge assembly 1 is opened relative to the staple anvil assembly 2, the cutter 6 is located at the position 6a at the proximal end of the nail cartridge assembly 1. By holding the operating handle, the cutter push rod 7 is enabled to drive the cutter 6 to move in the direction toward the distal end to the position 6b, and at this point, the staple cartridge assembly 1 gradually closes relative to the staple anvil assembly 2. After the jaw opening of the staple head is closed, the cutter 6 is driven by the cutter push rod 7 to continue moving in the direction toward the distal end to a distal end of the staple cartridge assembly 1, and thereby completing the firing of the stapler.

As shown in FIG. 6 to FIG. 11, the staple head also includes a stop member 5, the stop member 5 includes a first blocking portion 51, and a distal end of the cutter 6 is correspondingly provided with a second blocking portion 621. A distal end of the connecting member 3 is provided with a mounting portion 30 for mounting the stop member 5. When the staple cartridge assembly 1 is opened relative to the staple anvil assembly 2, the mounting portion 30 is at least partially located on a side of the second blocking portion 621 away from the axial center direction of the stapler, and the mounting portion 30 is at least partially located on a side in a transverse direction of the second blocking portion 621. The mounting portion 30 is provided with a mounting groove 31 opening in a direction toward the axial center of the stapler, and the stop member 5 at least partially extends into the mounting groove 31. The mounting portion 30 is internally provided with a mounting hole 34, the stop member 5 is provided with a pivoting portion 54, and the pivoting portion 54 is rotatably mounted in the mounting hole 34 of the mounting portion 30 via a pin shaft 36.

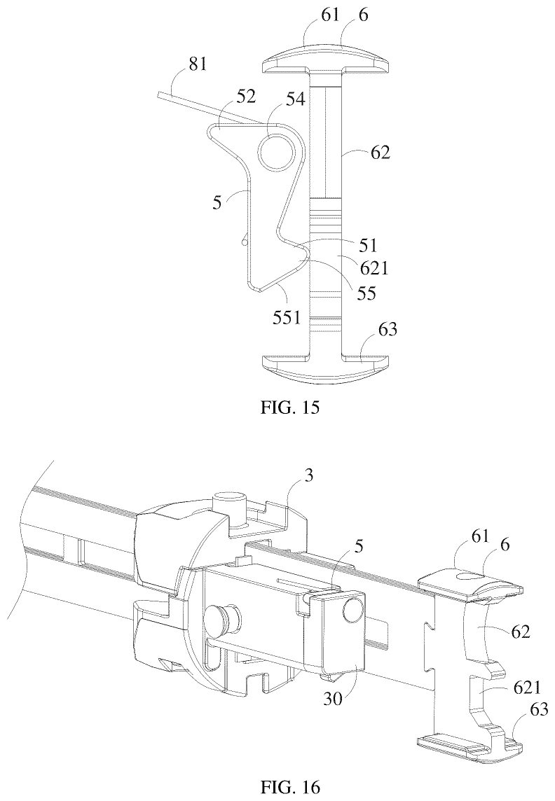

The stop member 5 has a first state and a second state, FIG. 10 and FIG. 11 show cooperation between the stop member 5 in the first state and the cutter 6, and FIG. 15 shows the cooperation between the stop member 5 in the second state and the cutter 6. As shown in FIG. 10 and FIG. 11, when the stop member 5 is in the first state, the first blocking portion 51 is located on a path where the second blocking portion 621 moves toward the distal end, and thereby blocking the cutter 6 to move toward the distal end, that is, the stapler cannot be fired, and thus accidental firing of the stapler is effectively avoided. As shown in FIG. 15, when an external force is applied to the stop member 5, the stop member 5 rotates relative to the connecting member 3 in a first direction to make the first blocking portion 51 move away from the axial center of the stapler, and the stop member 5 enters the second state. In the second state, the first blocking portion 51 disengages from the path where the second blocking portion 621 moves toward the distal end, and at this point, the cutter 6 is driven to move toward the distal end, and thus the stapler is normally fired. From the perspective of FIG. 11, the first direction is a clockwise direction.

The mounting groove 31 is internally provided with an elastic member which is configured to apply a biasing force to the stop member 5 to rotate the stop member 5 in a second direction, and the second direction is opposite to the first direction. The second direction is a counterclockwise direction from the perspective of FIG. 11. Therefore, when there is no an external force applied to the stop member 5, the stop member 5 does not rotate in the first direction, and the stop member 5 is remained in the first state under the action of the elastic member. As shown in FIG. 6, in this embodiment, the elastic member is a torsion spring 81 sleeved on the pin shaft 36. When an external force is applied to the stop member 5, the stop member 5 rotates in the first direction to enter the second state, the torsion spring 81 is driven to elastically deform. After the external force acting on the stop member 5 is removed, under the action of the restoring force of the elastic deformation of the torsion spring 81, the stop member 5 rotates in the second direction to return to the first state from the second state.

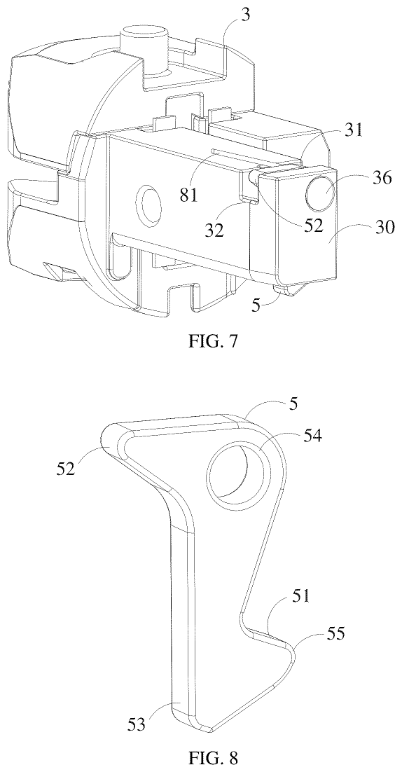

As shown in FIG. 7 to FIG. 10, the connecting member 3 includes a first limiting portion 32 and a third limiting portion 33, and the stop member 5 is provided with a second limiting portion 52 and a fourth limiting portion 53. Positions of the second limiting portion 52 and the fourth limiting portion 53 are adapted to the first limiting portion 32 and the third limiting portion 33, respectively. The second limiting portion 52 and the fourth limiting portion 53 are respectively located on two sides in a longitudinal direction of the pivoting portion 54, that is, they are located above and below the pivoting portion 54, respectively. The second limiting portion 52 protrudes in a direction away from the axial center of the stapler compared with the pivoting portion 54, and the second limiting portion 52 may also serve to reinforce an overall structural strength of the stop member 5. The first limiting portion 32 is a stepped portion that is adapted to a shape of the second limiting portion 52, and an extending direction of an upper surface of the stepped portion intersects with an extending direction of the third limiting portion 33. The first limiting portion 32 cooperates with the second limiting portion 52 to limit a rotation angle of the connecting member 3 in the second direction. When the stop member 5 rotates in the second direction under the action of the torsion spring 81, the first limiting portion 32 limits a rotation stroke of the second limiting portion 52 to prevent a rotation angle of the stop member 5 from being too large. The fourth limiting portion 53 is a side wall of the stop member 5 at a side which is away from the axial center of the stapler, and the third limiting portion 33 is an inner wall of the mounting groove 31 at a side which is away from the axial center of the stapler. In this embodiment, a surface of the first limiting portion 32 opposite to the second limiting portion 52 is substantially perpendicular to the longitudinal direction, and an extending direction of the third limiting portion 33 is substantially parallel to the longitudinal direction. The third limiting portion 33 cooperates with the fourth limiting portion 53 to limit the rotation angle of the stop member 5 in the first direction. When the stop member 5 rotates in the first direction under the action of an external force, the third limiting portion 33 limits a rotation stroke of the fourth limiting portion 53 to prevent the rotation angle of the stop member 5 from being too large. In another alternative embodiment, the mounting portion 30 of the connecting member 3 may not be provided with the mounting groove 31, but instead, the stop member 5 is rotatably mounted on the stop member 5 on either a side surface or a distal end surface of the mounting portion 30 directly, which also falls within the scope of protection of the present application.

In this embodiment, the switching of the stop member 5 from the first state to the second state is driven by a driving member 4. The driving member 4 is a firing block disposed on the staple cartridge assembly 1. A first driving portion 41 is provided on a proximal end of the driving member 4, and a side of the stop member 5 facing toward the axial center of the stapler is provided with a second driving portion 55 cooperating with the first driving portion 41. A first guiding surface 411 is provided on a side of the first driving portion 41 opposite to the second driving portion 55, and a second guiding surface 551 is provided on a side of the second driving portion 55 opposite to the first driving portion 41. The first guiding surface 411 is an inclined surface located above the first driving portion 41. In this embodiment, the first blocking portion 51 and the second driving portion 55 are integrally formed, that is, as shown in FIG. 11, a protruding block that protrudes toward the cutter 6 serves as both the first blocking portion 51 and the second driving portion 55. When the stop member 5 is in the first state, a side of the first blocking portion 51 facing toward the staple cartridge assembly 1 is provided with the second guiding surface 551, and at this point, the second guiding surface 551 is inclined relative to both the longitudinal direction and the axial direction.

As shown in FIG. 5 and FIG. 12 to FIG. 15, when the staple cartridge assembly 1 is opened relative to the staple anvil assembly 2, the driving member 4 is located on the proximal end of the staple cartridge assembly 1, but because the staple cartridge assembly 1 is located at a position away from the staple anvil assembly 2, the driving member 4 is located at the position 4a in FIG. 5, so that the driving member 4 will not contact with the stop member 5, and thus the stop member 5 is in the first state under the action of the torsion spring 81. The cutter 6 is located at the position 4a on the proximal end of the stop member 5, and there is a first axial distance between the second blocking portion 621 of the cutter 6 and the first blocking portion 51 of the stop member 5. When the cutter 6 moves a second distance in the direction toward the distal end to the position 6b, the staple cartridge assembly 1 gradually approaches the staple anvil assembly 2 to close the jaw opening, and the driving member 4 moves with the staple cartridge assembly 1 to the position 4b. Since the first distance is greater than the second distance, a distance between the proximal end of the driving member 4 and the distal side of the cutter 6 is a third distance in the axial direction at this point. The third distance is the distance between the position 4b and the position 6b, which is substantially equal to the first distance minus the second distance. In an alternative embodiment, the second distance may also be equal the first distance, at this time the proximal end of the driving member 4 abuts against the distal side of the cutter 6. As the driving member 4 moves from the position 4a to the position 4b, the first driving portion 41 of the driving member 4 moves toward the stop member 5 until it abuts against the second driving portion 55, and drives the stop member 5 to rotate in the first direction, so that the first blocking portion 51 moves in the direction away from the axial center of the stapler, and thus making the stop member 5 enter the second state.

The working principle of the stop member 5 in different states is specifically described below with reference to FIG. 5 and FIG. 10 to FIG. 17.

When the staple cartridge assembly 1 is opened relative to the staple anvil assembly 2, the driving member 4 is located at the position 4a on the proximal end of the staple cartridge assembly 1, so that it will not contact with the stop member 5. The cutter 6 is located at the position 6a on the proximal end of the staple cartridge assembly 1. As shown in FIG. 10 and FIG. 11, the driving member 4 does not apply an acting force to the stop member 5, under the action of the torsion spring 81, the stop member 5 is remained in the first state, and the first blocking portion 51 is located on the path where the second blocking portion 621 moves toward the distal end. The cutter 6 is located on the proximal end of the stop member 5, and there is the first distance between the second blocking portion 621 and the first blocking portion 51 of the stop member 5 in the axial direction.

When the staple head is placed in a surgical position, the jaw opening is required to be closed to clamp the tissue. At this point, by holding the operating handle, the transmission mechanism drives the cutter push rod 7 and the cutter 6 to move the second distance in the direction toward the distal end to the position 6b, the staple cartridge assembly 1 gradually approaches the staple anvil assembly 2 to close the jaw opening, and the driving member 4 moves with the staple cartridge assembly 1 to the position 4b. In this state, the third distance is formed between the proximal end of the driving member 4 and the distal side of the cutter 6. As shown in FIG. 12 and FIG. 14, during a closing process of the jaw opening, the first driving portion 41 of the driving member 4 abuts against the second driving portion 55 of the stop member 5 to drive the stop member 5 to rotate in the first direction, so that the first blocking portion 51 moves away from the axial center of the stapler, that is, it moves toward an outer side of the cutter 6, and then the stop member 5 moves to the second state shown in FIG. 15. During this process, the torsion spring 81 is compressed to generate elastic deformation, and the first blocking portion 51 disengages from the path that the second blocking portion 621 moves toward the distal end.

After the staple head is closed and the stop member 5 enters the second state, the operating handle is held again, so that the cutter push rod 7 and the cutter 6 is driven by the transmission mechanism to continue moving in the direction toward the distal end. The cutter 6 firstly moves to the position 6c to abut against the proximal end of the driving member 4, and then drives the driving member 4 located at the position 4b to move toward the distal end to the position 4c to push the staple out to suture the tissue, and the cutter 6 cuts the tissue, so that normal firing of the stapler is achieved. In the firing process of the stapler, the driving member 4 moves in the direction toward the distal end to enable the first driving portion 41 to disengage from the second driving portion 55, and the first stopping portion 51 of the stop member 5 abuts against the side wall of the connecting portion 62 of the cutter 6 such that the stop member 5 is remained in the second state. After the cutter 6 completely moves to the distal end of the stop member 5, as shown in FIG. 16, the first stopping portion 51 of the first blocking portion 51 abuts against a side surface of the cutter push rod 7 to remain the stop member 5 in the second state, and the torsion spring 81 is remained in a state with an elastic deformation amount.

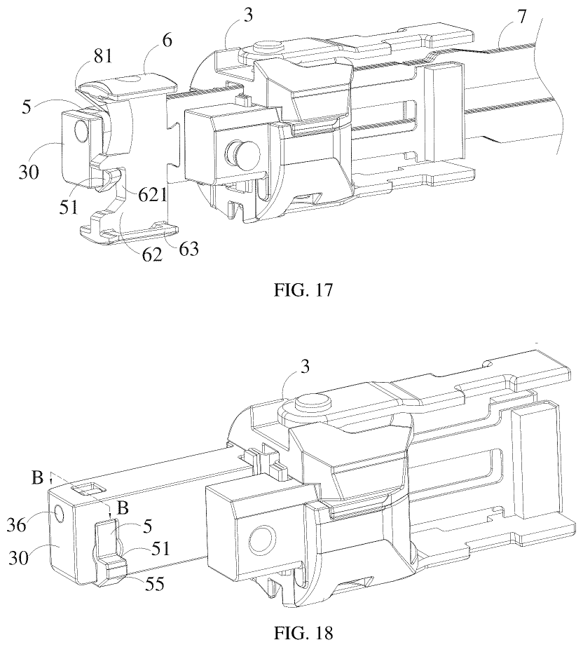

After the firing of the stapler is completed, under the holding action of the cutter push rod 7, the stop member 5 is still in the second state, at this time, the cutter 6 and the cutter push rod 7 are driven by the reset mechanism to reset towards the proximal end, while the driving member 4 still remains at the position 4c, and will not be reset to the proximal end of the staple cartridge assembly 1. In the process that the cutter 6 is reset from the distal end to the proximal end, the first blocking portion 51 of the stop member 5 still abuts against the side wall of the cutter push rod 7, and when the cutter 6 moves to the position where the stop member 5 is located, the first blocking portion 51 abuts against the side wall of the connecting portion 62 of the cutter 6 until the cutter 6 is completely reset to the proximal end of the first blocking portion 51. At this point, the first blocking portion 51 no longer abuts against the cutter 6 and the cutter push rod 7, and there is no driving force from the driving member 4. Therefore, under the action of the restoring force of the elastic deformation of the torsion spring 81, the stop member 5 rotates in the second direction, so that the first blocking portion 51 moves toward the axial center of the stapler, that is, it moves towards the inner side, thereby the first blocking portion 51 enters the path that the second blocking portion 621 moves toward the distal end again, and thus the stop member 5 enters the first state. At this point, if the operating handle is held to drive the cutter 6 to move toward the distal end to a position where the staple head is closed, since there is no driving member 4 at the proximal end of the staple cartridge assembly 1, so that the stop member 5 is still in the first state. If the operating handle is held to drive the cutter 6 move in the direction toward the distal end, as shown in FIG. 17, the second blocking portion 621 of the cutter 6 is blocked by the first blocking portion 51 of the stop member 5, thereby the cutter 6 cannot continue moving in the direction toward the distal end, so that the stapler cannot be fired, and thus secondary accidental firing when the staple cartridge 11 has not been replaced is effectively avoided.

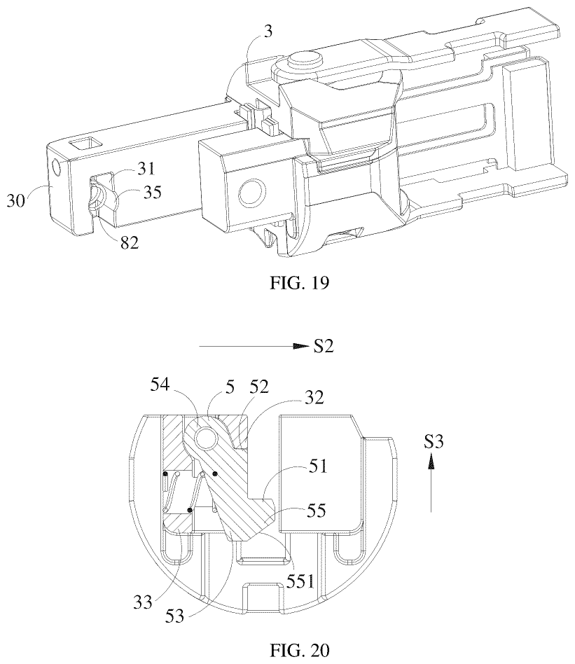

FIG. 18 to FIG. 20 show a structure that the stop member 5 cooperates with connecting member 3 according to a second embodiment of the present application. The difference between this embodiment and the first embodiment lies in that the structure which the connecting member 3 cooperates with the stop member 5 is different, and the structure which the elastic member cooperates with the stop member 5 is different. In this embodiment, the elastic member is a compression spring 82, and a compression spring channel 35 for accommodating the compression spring 82 is disposed inside the mounting groove 31. The compression spring 82 is movably disposed in the compression spring channel 35, and the compression spring channel 35 is arranged perpendicular to a side wall of the cutter 6. An axial width of the compression spring channel 35 is greater than an axial width of the mounting groove 31, so that the compression spring 82 will not fall out from the bottom of the mounting groove 31. Two ends of the compression spring 82 abut against a bottom wall of the compression spring channel 35 and an outer surface of the stop member 5, respectively. The second limiting portion 52 is a protruding portion located above the pivoting portion 54 and protruding toward an inner side relative to the pivoting portion 54, and the first limiting portion 32 is a stepped portion located on a bottom wall of the mounting groove 31 and shaped to match with the second limiting portion 52 to limit the rotation angle of the stop member 5 in the second direction. An upper surface of the stepped portion is substantially perpendicular to the longitudinal direction and intersects with an extending direction of the third limiting portion 33. The second limiting portion 52 may also reinforce the overall structural strength of the stop member 5. The third limiting portion 33 is an inner wall located below the compression spring channel 35, and the fourth limiting portion 53 is a side wall located below the pivoting portion 54 and located on an outer side of the stop member 5. When an external force is applied to the stop member 5, the stop member 5 rotates in the first direction to enter the second state, the stop member 5 compresses the compression spring 82 to elastically deform, and the third limiting portion 33 cooperates with the fourth limiting portion 53 to limit the rotation angle of the stop member 5 in the first direction. After the external force acting on the stop member 5 is removed, under the action of a restoring force of the elastic deformation of the compression spring 82, the stop member 5 rotates in the second direction to enter the first state. Other structures in the second embodiment are the same as the corresponding structures in the first embodiment, and will not be described in detail herein again. The structure in the second embodiment may also be combined with the first embodiment, for example, the torsion spring 81 arranged in the mounting groove 31 in the first embodiment is directly replaced with the compression spring 82, the structure of the stop member 5 remains unchanged, or the matching structure of the mounting groove 31 and the stop member 5 in the first embodiment is replaced with the structure in the second embodiment, while retaining the torsion spring 81 as the elastic member. In this embodiment, the torsion spring 81 in the first embodiment is replaced with the compression spring 82, a gap between the stop member 5 and the connecting member 3 is reduced while increasing the connection strength, and thereby preventing the stop member 5 from wobbling during use.

In the foregoing embodiments, taking the first jaw as the staple cartridge assembly and the second jaw as the staple anvil assembly as an example for illustrating, the staple anvil assembly is fixed relative to the connecting member, the staple cartridge assembly is rotatable relative to the connecting member, and the driving member is disposed on the staple cartridge assembly. In other alternative embodiments, the first jaw may be configured as the staple anvil assembly and the second jaw as the staple cartridge assembly, and with the driving member being disposed on the staple anvil assembly. In such construction, the staple cartridge assembly is fixed relative to the connecting member, and the staple anvil assembly is rotatable relative to the connecting member. Before the staple head is closed, the driving member in the staple anvil assembly is away from the stop member without abutting against the second driving portion, and when the staple head is closed and the driving member is located on the proximal end of the staple anvil assembly, the driving member abuts against the second driving portion to drive the stop member to enter the second state.

In the foregoing embodiment, taking the first blocking portion and the second driving portion being integrally formed as an example for description. In other alternative embodiments, the first blocking portion and the second driving portion may also be two components, for example, two protrusions are disposed on an inner side of the stop member, and the second driving portion is located below the first blocking portion. When the staple cartridge assembly rotates to gradually close the jaw opening, the first driving portion firstly abuts against the second driving portion to drive the stop member to rotate in the first direction.

In the above embodiments, two exemplary implementations of the elastic member are provided with a torsion spring and a compression spring. In other alternative implementations, the elastic member may also adopt other structures. For example, the compression spring in the second embodiment may be replaced with a spring plate an elastic pad, or the like. Alternatively, the elastic member may also be a tension spring connected between the inner side of the stop member and a position of the staple cartridge frame close to the second cutter groove, and in the first state, the biasing force of the tension spring to the stop member is a pulling force that pulls the first blocking portion toward the inner side.

The foregoing content is a further detailed description of the present application with reference to specific preferred embodiments, and it cannot be determined that the specific implementation of the present application is limited to these descriptions. For a person of ordinary skill in the art to which this application belongs, several simple inferences or substitutions may still be made without departing from the inventive concept of the present application, and all of them should be regarded as falling within the protection scope of this application.

What is claimed is:

1. A staple head for a surgical stapler, comprising:
a first jaw and a second jaw arranged opposite to each other;
a connecting member, connected to proximal ends of the first jaw and the second jaw;
a stop member rotatably mounted on the connecting member and comprising a first blocking portion; and
a firing mechanism movable along an axial direction of the surgical stapler and comprising a second blocking portion; wherein
when the stop member is in a first state, the first blocking portion is located on a path where the second blocking portion moves toward a distal end; and when an external force is applied to the stop member, the stop member rotates relative to the connecting member in a first direction such that the first blocking portion moves away from an axial center of the stapler, the stop member enters a second state, and the first blocking portion disengages from the path where the second blocking portion moves toward the distal end.

2. The staple head according to claim 1, wherein a distal end of the connecting member is provided with a mounting portion for mounting the stop member; when the first jaw and the second jaw are in an open state, the mounting portion is at least partially located on a side of the second blocking portion away from an axial center of the stapler; and a first distance is set between the first blocking portion and the second blocking portion along the axial direction of the surgical stapler.

3. The staple head according to claim 2, wherein the stop member further comprises a pivoting portion rotatably mounted on the mounting portion via a pin shaft; and when the stop member is in the first state, the first blocking portion protrudes in a direction toward the axial center of the stapler relative to the pivoting portion.

4. The staple head according to claim 2, further comprising a driving member disposed on the first jaw and movable along the axial direction of the stapler, wherein the first jaw is rotatable relative to the connecting member; and
when the driving member is located at the proximal end of the first jaw and the first jaw rotates to be relatively closed with the second jaw, the driving member abuts against the stop member and drives the stop member to enter the second state from the first state.

5. The staple head according to claim 4, wherein when the stop member enters the second state from the first state, the firing mechanism moves toward the distal end for a second distance, and the first distance is greater than the second distance.

6. The staple head according to claim 4, wherein when the stop member enters the second state from the first state, the firing mechanism moves a second distance in the direction toward the distal end, and the first distance is equal to the second distance.

7. The staple head according to claim 4, wherein a first driving portion is provided on a proximal end of the driving member, and a second driving portion cooperating with the first driving portion is provided on a side of the stop member facing toward the axial center of the stapler.

8. The staple head according to claim 7, wherein a side of the first driving portion opposite to the second driving portion is provided with a first guiding surface, and a side of the second driving portion opposite to the first driving portion is provided with a second guiding surface.

9. The staple head according to claim 8, wherein the first blocking portion and the second driving portion are integrally formed; and when the stop member is in the first state, a side of the first blocking portion facing toward the first jaw is provided with the second guiding surface.

10. The staple head according to claim 1, wherein the firing mechanism comprises a cutter and a cutter push rod, a distal end of the cutter is provided with the second blocking portion, and a proximal end of the cutter is connected to the cutter push rod.

11. The staple head according to claim 10, wherein when the stop member is in the second state and the firing mechanism moves toward the distal end the stop member at least partially abuts against a side surface of the cutter push rod to be remained in the second state.

12. The staple head according to claim 3, further comprising an elastic member configured to apply a biasing force to the stop member to drive the stop member to rotate in a second direction opposite to the first direction.

13. The staple head according to claim 12, wherein the mounting portion is provided with a mounting groove, and an opening of the mounting groove faces toward the axial center of the stapler, and the stop member at least partially enters into the mounting groove.

14. The staple head according to claim 13, wherein the elastic member is a torsion spring disposed at the pin shaft.

15. The staple head according to claim 13, wherein the elastic member is a compression spring disposed between the stop member and an inner wall of the mounting groove.

16. The staple head according to claim 15, wherein a compression spring channel is provided inside the mounting groove, the compression spring is movably disposed in the compression spring channel, and the compression spring channel is arranged perpendicular to a side wall of the firing mechanism.

17. The staple head according to claim 16, wherein an axial width of the compression spring channel is greater than an axial width of the mounting groove.

18. The staple head according to claim 13, wherein the connecting member comprises a first limiting portion, and the mounting groove is provided with a second limiting portion; and the first limiting portion cooperates with the second limiting portion to limit a rotation angle of the connecting member in the second direction.

19. The staple head according to claim 13, wherein the connecting member further comprises a third limiting portion, and the stop member comprises a fourth limiting portion; and the third limiting portion cooperates with the fourth limiting portion to limit a rotation angle of the connecting member in the first direction.

20. A surgical stapler, comprising the staple head according to claim 1.
